(19) United States
(12) Patent Application Publication
(10) Pub. No.: US 2006/0054276 A1
(43) Pub. Date: Mar. 16, 2006

(54) Use of an acidic aqueous solution of a bioadhesive polyphenolic protein as an adhesive or coating

(76) Inventor: Magnus Qvist, Alingsas (SE)

Correspondence Address: ALBIHNS STOCKHOLM AB, BOX 5581, LINNEGATAN 2, SE-114 85 STOCKHOLM; SWEDENn, STOCKHOLM, SE

(21) Appl. No.: 10/520,023

(22) PCT Filed: Jun. 24, 2003
(86) PCT No.: PCT/SE03/01088

§ 371 Date: Dec. 30, 2004

(30) Foreign Application Priority Data
Jul. 2, 2002 (SE) 02020659
Jul. 2, 2002 (US) 60392971

(51) Int. Cl. C07K 14/435; C09J 5/04

(52) U.S. Cl. 156/317; 530/350

(57) ABSTRACT

The present invention pertains to the use of an acidic aqueous solution of a bioadhe-sive polyphenolic protein, derived from a byssus-forming mussel, for attaching two surfaces to each other or coating a surface, which acidic solution has a pH of 4 or less and in which the concentration of the bioadhesive protein is between 10-250 mg/ml. The acidic aqueous solution of the polyphenolic protein is added directly as a sole component to the surfaces to be attached to each other or the surface to be coated.

The present invention pertains to the direct use of an acidic aqueous solution of a bioadhesive protein for attaching two surfaces to each other or coating a surface.

### BACKGROUND OF THE INVENTION

Attachment of different structures is crucial in a wide variety of processes. However, this is frequently associated with problems of different nature depending on what structures are to be attached.

Areas that are particularly troublesome are adhesion in the medical field, and attachment of components of very small size, such as in the micro- and nano-techniques. In the medical field, examples of when adhesives have to be used to adhere biological material include repair of lacerated or otherwise damaged organs, especially broken bones and detached retinas and corneas. Dental procedures also often require adhesion of parts to each other, such as during repair of caries, permanent sealants and periodontal surgery. It is very important in biomedical applications of an adhesive and coating composition to use bioacceptable and biodegradable components, which furthermore should not per se or due to contamination induce any inflammation or toxic reactions. In addition, the adhesive has to be able to attach structures to each other in a wet environment.

In the electronic industry, a particular problem today is that the components that are to be attached to each other often are of very small size, and the amount of adhesive that is possible to use is very small. Adhesives that provide high adhesive strength even with minor amounts of adhesive are therefore required. Also for non-medical uses, an adhesive that is non-irritating, non-allergenic, non-toxic and environmentally friendly is preferred. However many of the commonly used adhesives induce toxic reactions in the user, for example due to their contents of organic solvent.

Polyphenolic proteins, preferentially isolated from mussels, are known to act as adhesives. Examples of such proteins can be found in e.g. U.S. Pat. No. 4,585,585. Their wide use as adhesives has been hampered by problems related to the purification and characterisation of the adhesive proteins in sufficient amounts. Also, mostly when using the polyphenolic proteins as adhesives the pH has had to be raised to neutral or slightly basic in order to facilitate oxidation and curing of the protein. However, this curing is slow and results in poor adhesive strength and therefore oxidisers, fillers and cross-linking agents are commonly added to decrease the curing time and obtain a stronger adhesive. In addition, in an earlier study (EP-A-244 688) the adhesive strength using, as a sole component without raising the pH, an acidic solution of MAP (5 mg/ml in 5% acetic acid) was demonstrated to be poor, compared to when a filler protein was added to the composition before adhesion (2.5 mg/ml each of MAP and casein).

Mussel adhesive protein (MAP) is formed in a gland in the foot of byssus-forming mussels, such as the common blue mussel (Mytilus edulis). The molecular weight of MAP from Mytilis edulis is about 130.000 Dalton and it has been disclosed to consist of 75-80 closely related repeated peptide sequences. The protein is further characterised by its many epidermal growth factor like repeats. It has an unusual high proportion of hydroxy-containing amino acids such as hydroxyproline, serine, threonine, tyrosin, and the uncommon amino acid 3,4-dihydroxy-L-phenylalanine (Dopa) as well as lysine. It may be isolated either from natural sources or produced biotechnologically. U.S. Pat. No. 5,015,677 as well as U.S. Pat. No. 4,585,585 disclose that MAP has very strong adhesive properties after oxidation and polymerisation, e.g. by the activity of the enzyme tyrosinase, or after treatment with bifunctional reagents.

MAP is previously known to be useful as an adhesive composition e.g. for ophthalmic purposes. Robin et al., Refractive and Corneal Surgery, vol. 5, p. 302-306, and Robin et al., Arch. Ophthalmol., vol. 106, p. 973-977, both disclose MAP-based adhesives comprising an enzyme polymiser. U.S. Pat. No. 5,015,677 also describes a MAP-based adhesive containing a cross-linking agent and optionally a filler substance and a surfactant. Preferred cross-linking agents according to U.S. Pat. No. 5,015,677 are enzymatic oxidising agents, such as catechol oxidase and tyrosinase, but sometimes also chemical cross-linking agents, such as glutaraldehyde and formaldehyde can be used. Examples of fillers are proteins, such as casein, collagen and albumin, and polymers comprising carbohydrate moieties, such as chitosan and hyaluronan. U.S. Pat. No. 5,030,230 also relates to a bioadhesive comprising MAP, mushroom tyrosinase (cross-linker), SDS (sodium dodecyl sulfate, a surfactant) and collagen (filler). The bioadhesive is used to adhere a cornea prosthesis to the eye wall.

EP-A-343 424 describes the use of a mussel adhesive protein to adhere a tissue, cell or another nucleic acid containing sample to a substrate during nucleic acid hybridisation conditions, wherein the mussel adhesive protein, despite the harsh conditions encountered during the hybridisation, provided adherence. U.S. Pat. No. 5,817,470 describes the use of mussel adhesive protein to immobilise a ligand to a solid support for enzyme-linked immunoassay. Mussel adhesive protein has also been used in cosmetic compositions to enhance adherence to nails and skin (WO 88/05654).

A major problem associated with known MAP-based bioadhesive compositions, despite the superior properties of MAP per se, is that some constituents, in particular commonly used cross-linking agents, can harm and/or irritate living tissue and cause toxic and immunological reactions. Chemical crosslinking agents, such as glutaraldehyde and formaldehyde, are generally toxic to humans and animals, and it is highly inappropriate to add such agents to a sensitive tissue, such as the eye. Enzymes, such as catechol oxidase and tyrosinase, are proteins, and proteins are generally recognised as potential allergens, especially in case they originate from a species other than the patient. Because of their oxidising and hydrolysing abilities, they can also harm sensitive tissue.

Therefore, there is still a need for adhesive compositions, both for medical and other applications, that provide strong adhesion with small amounts of adhesive, that are simple to use and that do not cause toxic and allergic reactions.

### SUMMARY OF THE INVENTION

The present invention pertains to the use of an acidic aqueous solution of a bioadhesive polyphenolic protein, derived from a byssus-forming mussel, for attaching two surfaces to each other or coating a surface, which acidic solution has a pH of 4 or less and in which the concentration of the bioadhesive protein is between 10-250 mg/ml. The use of this acidic solution of the bioadhesive protein as a sole component avoids the addition of additional components to effect adhesion and therefore the process of adhesion is simplified and the risk of causing allergy and/or irritation due to the additional components added is decreased. The composition is therefore well-suited for medical application. Also, the adhesive strength obtained is high, even with small amounts of adhesive, and the composition is therefore also preferably used when only small amounts of adhesive can be applied to surfaces to be joined or coated. The composition of the present invention is also suitable for use in wet environments.

### DEFINITIONS

As disclosed herein, the terms “polyphenolic protein”, “mussel adhesive protein” or “MAP” relates to a bioadhesive protein derived from byssus-forming mussels or which is recombinantly produced. Examples of such mussels are mussels of the genera Mytilus, Geukensia, Aulacomya, Phragmatopoma, Dreissenia and Brachiodontes. Suitable proteins have been disclosed in a plurality of publications, e.g. U.S. Pat. No. 5,015,677, U.S. Pat. No. 5,242,808, U.S. Pat. No. 4,585,585, U.S. Pat. No. 5,202,236, U.S. Pat. No. 5,149,657, U.S. Pat. No. 5,410,023, WO 97/34016, and U.S. Pat. No. 5,574,134, Vreeland et al., J. Physiol., 34: 1-8, and Yu et al., Macromolecules, 31: 4739-4745. They comprise about 30-300 amino acid residues and essentially consist of tandemly linked peptide units comprising 3-15 amino acid residues, optionally separated by a junction sequence of 0-10 amino acids. A characteristic feature of such proteins is a comparatively high amount of positively charged lysine residues, and in particular the unusual amino acid DOPA (L-3,4-dihydroxyphenylalanine). A polyphenolic protein suitable for use in the present invention has an amino acid sequence in which at least 3% and preferably 6-30% of the amino acid residues are DOPA. A few examples of typical peptide units are given below. However, it is important to note that the amino acid sequences of these proteins are variable and that the scope of the present invention is not limited to the exemplified subsequences below, as the skilled person realises that bioadhesive polyphenolic proteins from different sources, including recombinantly produced, can be regarded as equivalent:
a) Val-Gly-Gly-DOPA-Gly-DOPA-Gly-Ala-Lys
b) Ala-Lys-Pro-Ser-Tyr-diHyp-Hyp-Thr-DOPA-Lys
c) Thr-Gly-DOPA-Gly-Pro-Gly-DOPA-Lys
d) Ala-Gly-DOPA-Gly-Gly-Leu-Lys
e) Gly-Pro-DOPA-Val-Pro-Asp-Gly-Pro-Tyr-Asp-Lys
f) Gly-Lys-Pro-Ser-Pro-DOPA-Asp-Pro-Gly-DOPA-Lys
g) Gly-DOPA-Lys
h) Thr-Gly-DOPA-Ser-Ala-Gly-DOPA-Lys
i) Gln-Thr-Gly-DOPA-Val-Pro-Gly-DOPA-Lys
j) Gln-Thr-Gly-DOPA-Asp-Pro-Gly-Tyr-Lys
k) Gln-Thr-Gly-DOPA-Leu-Pro-Gly-DOPA-Lys

The term “surface” is to be interpreted broadly and may comprise virtually any surface. The choice of surface is not critical to the present invention. Examples of surfaces for which the invention are specially suitable for include non-biological surfaces such as glass, plastic, ceramic and metallic surfaces etc., and biological surfaces, comprising wood and different tissues such as skin, bone, teeth, the eye, cartilage, etc.

By acidic aqueous solution is meant an aqueous solution comprising an organic or inorganic acid.

### DETAILED DESCRIPTION OF THE INVENTION

The present invention describes the use of a polyphenolic bioadhesive composition to attach two surfaces to each other or coating a surface. The compositions provided in the invention can in principle be used to attach any surfaces to each other or to coat any surface. However, the compositions according to the present invention are particularly useful when adhesive or coating compositions are needed that are non-toxic, non-irritating or non-allergenic, since the only mandatory component is the bioadhesive protein in itself and this has a low risk of causing such reaction. The use of a bioadhesive composition described in the present invention allows very small amounts of adhesive to be used while still achieving a strong adhesion. Therefore the use of the composition of the present invention is particularly useful when only small amounts of adhesive can be used. Further advantages with the use of the composition provided in the present invention are their water solubility, the avoidance of organic solvents commonly used in adhesive or coating compositions, and that they are biologically produced and harmless to the environment.

The only mandatory component of the present invention is the polyphenolic protein itself provided in an acidic solution, for example the same acidic solution that is used for storage of the protein. Previously when polyphenolic proteins have been used, it has been thought to be necessary to add additional components, such as fillers and oxidising agents and/or raise the pH to neutral or slightly basic, in order to achieve strong enough adhesive strength. The present inventor has shown that a very strong adhesion, comparable to the adhesive strength provided using the commonly used MAP compositions, can be provided employing a concentrated acidic MAP-solution directly. Therefore, since no additional components have to be added to the MAP-solution before its use, the process of adhesion is simplified over earlier uses of bioadhesive proteins. Also due to the simple composition of the adhesive, the risks of irritation and/or allergy that have been common with earlier uses of bioadhesive polyphenolic proteins is avoided.

According to the present invention the acidic solution of the MAP-protein is applied, as a sole component, to at least one of the surfaces, which are to be attached to each other, before the surfaces are joined, or added to the surface to be coated. The composition of the invention was demonstrated to cure both in dry and wet environments. As can be seen in the appended Examples the curing time can be as short as 1 min.

The concentration of the MAP-solution of the present invention is 10-250 mg/ml. Preferably the concentration of the MAP-solution is 10-150 mg/ml. More preferably the MAP-concentration is 30-100 mg/ml and most preferably 40-80 mg/mL. It is important that the concentration of the MAP-solution is at least 10 mg/ml, since earlier experiments have shown a poor adhesive strength using a 5 mg/ml MAP-solution in 5% acetic acid (EP-A-244 688), if no additional components were added to effect curing.

The MAP protein of the present invention is provided in an acidic aqueous solution with a pH of 4 or less. However, a pH of 3 or less was also unexpectedly found to result i high adhesive strengths. Even more surprisingly at pH of 2.5 or less was found to result in high adhesive strengths. Acids suitable for the present invention include both inorganic acids, such as hydrochloric acid and phosphoric acid, and organic acids, such as citric acid, ascorbic acid, and acetic acid. One preferred object of the present invention is to provide an adhesive or coating composition for medical applications, e.g. for attaching biological and non-biological components to biological structures, an object for which the MAP-protein in itself is well suited, since it is non-toxic and biodegradable. However, the components commonly added to MAP-compositions in order to obtain cross-linking and oxidation (chemical and/or enzymatic crosslinkers and oxidising agents) of the composition can lead to irritation and allergic reactions and those MAP-compositions are therefore not optimal for medical applications. Due to the lack of such components in the present invention, the compositions of the present invention are particularly suitable for attachment of biological surfaces to each other or to other, non-biological, materials. Also, since only small amounts of the adhesive composition of the present invention is required, while still providing high adhesive strengths, the composition of the present invention is particularity suitable for medical applications where often only small amounts of adhesives can be applied to surfaces to be adhered to each other or surfaces to be coated. For the above reasons, the use of the composition of the present invention is particularily suitable for adhesion of corneas, tendons, tissues during surgical operation etc. For the above reasons, the compositions of the present invention are also particularly useful for coating of materials used in medical applications or biological tissues.

Due to the very high adhesive strength provided with very small amounts of the compositions of the present invention, one preferred field of application for which the compositions are particularly suitable is for attachment of non-biological surfaces such as glass, plastic, ceramic and metallic surfaces. This is particularly useful within the electronic micro- and nano-techniques, optics, etc. for adhesion or coating of components in, for example, biosensors, microchips, solar cells, mobile phones, etc., since for these applications only minute amounts of adhesive can be used. The compositions of the present invention are also suitable for coating of non-biological surfaces.

The adhesive compositions of the present invention are also useful for attachment of cells, enzymes, antibodies and other biological specimen to surfaces.

### EXAMPLE 1

In order to determine the adhesive strength using the compositions of the present invention, the adhesive strength between non-biological material (glass plates, 75×25×2 mm) and biological tissue (muscle from cattle and pig) was determined. The aqueous, acidic MAP-solution with varying concentrations (see Table 1) in 0.01 M citric acid (pH ca 2.3) was applied to one of the surfaces that were to be attached to each other before joining the two surfaces and fixing them with a clip. The samples were thereafter allowed to cure for different time periods and under different conditions before the adhesive strength was determined using a digital spring balance (Milo) by attaching either the glass plate or the biological tissue to the balance and thereafter stretching until the glass plate and biological tissue were detached from each other. The adherence surfaces were in most cases 0.2-0.4 cm², with a variation from 0.1 to 0.8 cm². As can be seen in the results in Table 1 the adhesive strength is not weakened when the samples are allowed to cure under wet conditions, even though no cross-linking agent is employed.

TABLE 1
Adhesive strength achieved between biological and non-biological surfaces using the
MAP-composition of the present invention.

| | MAP | MAP | | | |
|---|---|---|---|---|---|
| | Concentration | Amount | Acid | | |
| Sample | (mg/ml) | (μg) | (concentration) | Curing conditions | Adhesive strength (g) |
| 1 | 23 | 69 | Citric acid (0.01 M) | 24 h in water at 4° C. | 40 |
| 2 | 25 | 75 | Citric acid (0.01 M) | 24 h in water at 4° C. | 45 |
| 3 | 20 | 60 | Citric acid (0.01 M) | 1 h in water at 35° C. | 60 |
| 4 | 20 | 60 | Citric acid (0.01 M) | 1 h in water at 35° C. | 40 |
| 5 | 24 | 72 | Citric acid (0.01 M) | 1 min under dry condi- | 40 |
| | | | | tions | |

### EXAMPLE 2

In order to determine the adhesive strength using the compositions of the present invention, the adhesive strength between biological tissue (muscle from cattle and pig) was determined. The acidic MAP-solution (see Table 2) in 0.01 M citric acid (pH ca 2.3) was applied to one of the surfaces that were to be attached to each other before joining the two surfaces and fixing them with a clip. The samples were thereafter allowed to cure under water at 35° C. before the adhesive strength was determined using a digital spring balance (Milo) by attaching one of the two parts of biological tissue to the balance and thereafter stretching until the biological tissues were detached from each other. The adherence surfaces were in most cases 0.2-0.4 cm², with a variation from 0.1 to 0.8 cm².

TABLE 2
Adhesive strength achieved between biological surfaces using the MAP-composition of
the present invention.

| | MAP | MAP | | | |
|---|---|---|---|---|---|
| | Concentration | Amount | Acid | | |
| Sample | (mg/ml) | (μg) | (concentration) | Curing conditions | Adhesive strength (g) |
| 1 | 20 | 50 | Citric acid (0.01 M) | 1 hour in water at 35° C. | 100 |
| 2 | 18 | 45 | Citric acid (0.01 M) | 1 hour in water at 35° C. | 120 |

### EXAMPLE 3

To determine the adhesive strength achieved between two non-biological surfaces, two glass plates (ca 75×25×1.5 mm) were attached to each other by placing a droplet of acidic MAP-solution on one of the glass plates, placing the other glass plate on top of the first and fixing the two glass plates to each other using a clip. The concentrations and amounts of the MAP-solutions employed are specified in Table 3 below, as is the acid, and its concentration, that is used for each specific experiment. The pH-values for the different acids employed were as follows: 0.05 M citric acid: pH ca 1.8; 0.01 M citric acid: pH ca 2.3; 0.2 M acetic acid: pH ca 2.3; 0.014 M ascorbic acid: pH 2.9; 0.05 M HCl: pH ca 1.0; and 0.05 M H₃PO₄: pH ca 1.4. The samples were left for 24 hours at room temperature before determining adhesive strength. The adhesive strength was determined by measurement of shear strength (see Table 3) employing conventional techniques. The adhesive area varied between 0.3-1.0 cm². As a comparison the adhesive strength employing standard epoxy glue was determined. Use of 10 mg of this to the glass plates in a similar fashion as described above resulted in an adhesive strength of 380 N.

TABLE 3
Adhesive strength achieved between non-biological surfaces using the MAP-composition
of the present invention.

| | MAP | MAP | Acid | Adhesive strength |
|---|---|---|---|---|
| Sample | Concentration (mg/ml) | Amount (μg) | (concentration) | (N) |
| 1 | 24 | 48 | Citric acid (0.01 M) | 226 |
| 2 | 42 | 42 | Citric acid (0.05 M) | 290 |
| 3 | 42 | 84 | Citric acid (0.05 M) | 430 |
| 4 | 39 | 117 | Citric acid (0.05 M) | 401 |
| 5 | 39 | 117 | Citric acid (0.05 M) | 437 |
| 6 | 42 | 42 | Citric acid (0.05 M) | 350 |
| 7 | 27 | 54 | Acetic acid (0.2 M) | >240 |
| 8 | 28 | 56 | Ascorbic acid (0.014 M) | 353 |
| 9 | 28 | 56 | Ascorbic acid (0.014 M) | 328 |
| 10 | 23 | 46 | HCI (0.05 M) | >270 |
| 11 | 25 | 50 | H₃PO₄ (0.05 M) | 237 |

1. A composition for attaching two surfaces to each other or for coating a surface, consisting of:
an acidic aqueous solution of a bioadhesive polyphenolic protein derived from a byssus-forming mussel which protein comprises 30-300 amino acids and consists essentially of tandemly linked peptide repeats comprising 3-15 amino acid residues, wherein
at least 3% of the amino acid residues of said bioadhesive polyphenolic protein are L-3,4-dihydroxyphenylalanine;
the pH of said acidic solution is 4 or less; and
the concentration said bioadhesive polyphenolic protein is 10-250 mg/ml.

2. A composition according to claim 1, wherein the pH of the acidic solution is 3 or less.

3. A composition according to claim 1, wherein the pH of the acidic solution is 2.5 or less.

4. A composition according to claim 1, wherein the acidic solution comprises an organic acid.

5. A composition according to claim 1, wherein the acidic solution comprises an inorganic acid.

6. A composition according to claim 1, wherein the acidic solution comprises an acid chosen from the group consisting of citric acid, acetic acid, and ascorbic acid.

7. A composition according to claim 1, wherein the concentration of the bioadhesive polyphenolic protein is in the range of 10-150 mg/ml.

8. A composition according to claim 1, wherein at least one of the surfaces to be attached or the surface to be coated is a biological surface.

9. Use of a composition according to claim 1, wherein at least one of the surfaces to be attached or the surface to be coated is a non-biological surface.

10. A composition according to claim 1, wherein 6-30% of the amino acid residues of said bioadhesive polyphenolic protein are L-3,4-dihydroxyphenylalanine.
